(19) United States
(12) Patent Application Publication
(10) Pub. No.: US 2022/0144740 A1
(43) Pub. Date: May 12, 2022

(54) BIODEGRADABLE SURFACTANT FOR HARD SURFACE CLEANERS

(71) Applicant: Dow Global Technologies LLC, Midland, MI (US)

(72) Inventors: Wanglin Yu, Pearland, TX (US); Bing Liang, Shanghai (CN); Paula A. Cameron, Pearland, TX (US)

(73) Assignee: Dow Global Technologies LLC, Midland, MI (US)

(21) Appl. No.: 17/438,531

(22) PCT Filed: May 30, 2019
(86) PCT No.: PCT/CN2019/089374

§ 371 Date: Sep. 13, 2021

(51) Int. Cl. C07C 43/11; C11D 1/722; C11D 11/00; C11D 3/00

(52) U.S. Cl. CPC C07C 43/11; C11D 3/0026; C11D 11/0029; C11D 1/722

(57) ABSTRACT

Provided is a surfactant of structure (I), wherein m is a value in a range of 3 to 10, n is a value in a range of 3 to 20 and z is a value in a range of 1 to 3. Said surfactant is useful as a biodegradable low foaming surfactant.

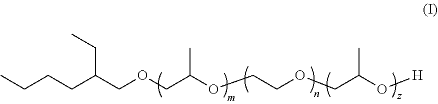

### BACKGROUND

### Field of the Invention

The present disclosure generally relates to surfactants, and more specifically, to biodegradable surfactants for hard surface cleaning.

### INTRODUCTION

Low foaming non-ionic surfactants may be useful in detergent and rinse aid products as hard surface cleaners. Such detergent and rinse aid products may be used in automatic dishwashers, metal cleaning, bottle cleaning, floor cleaning, window cleaning, and the cleaning in food and beverage processing. Biodegradable low foaming non-ionic surfactant are particularly desirable in order to avoid long-term impact on the environment. Examples of low foaming biodegradable non-ionic surfactants are known, but they have some technical limitations in order to achieve biodegradability.

U.S. Pat. Nos. 3,956,401 and 4,317,940 each describe a triblock copolymer of oxypropylene and oxyethylene. Specifically, U.S. Pat. Nos. 3,956,401 and 4,317,940 disclose an oxypropylene-oxyethylene-oxypropylene triblock copolymers prepared with a linear initiator in order to produce a linear aliphatic hydrocarbon on an oxypropylene end of the copolymer. The reason a linear hydrocarbon group is important in these references is that branching in a surfactant detrimentally affects biodegradability. For example, U.S. Pat. Nos. 3,956,401 and 4,317,940 each teach that “the biodegradability of the product is detrimentally affected by branching.” Therefore, to achieve biodegradability, the surfactants are prepared using linear alcohols as initiators. The detrimental effect of branching in biodegradability is further affirmed in a study of ethoxylate polymers that concluded that polymers initiated with single or multiple-branched alcohols did not show a significant degradation while significant degradation was observed to ethoxylates with linear alcohols and iso-alcohol. (See, M. T. Muller, M. Siegfried and Urs Bauman; “Anaerobic Degradation and Toxicity of Alcohol Ethoxylates in Anaerobic Screening Test Systems”, presented at 4^{th} World Surfactants Congress, 1996).

In addition to the detrimental affect of branching in the alcohols, GB294536A teaches that the relative placement of oxypropylene and oxyethylene groups on the surfactant are relevant to biodegradability. For example, GB294536A discloses the placement of oxypropylene groups adjacent to an alkyl group and the use of terminal oxyethylene groups to build a nonionic surfactant that is highly biodegradable. However, when the oxyethylene and oxypropylene groups are reversed and the oxypropylene groups are terminal, the surfactant exhibits a low degree of biodegradability. As such, GB294536A suggests the terminal oxypropylene groups detrimentally affect the biodegradability of surfactants.

The size of the oxypropylene and oxyethylene portions of the surfactant affect the properties of the surfactant. U.S. Ser. No. 10/150,936 describes an oxypropylene-oxyethylene-oxypropylene triblock copolymer that contains a branched alcohol. The experimental data of U.S. Ser. No. 10/150,936 demonstrates degradation in antifoaming and cleaning performance with decreasing size of the terminal oxypropylene end block. In fact, U.S. Ser. No. 10/150,936 discloses that the oxypropylene-oxyethylene-oxypropylene triblock with the highest foaming and least cleaning ability had a 5-9-5 triblock group size. As such, U.S. Ser. No. 10/150,936 suggests that antifoaming and cleaning performance decrease with decreasing size of terminal oxypropylene units.

In view of the detrimental affects on biodegradability, antifoaming and cleaning performance associated with the presence of terminal oxypropylene end groups and their decreasing size, it would be unexpected to discover a surfactant that has a branched alkyl end group and an alkoxylated portion with 1-5 oxypropylene end groups which exhibits good cleaning, biodegradability and antifoaming performance.

### SUMMARY OF THE DISCLOSURE

The present disclosure provides an unexpected biodegradable low foaming non-ionic surfactant that has a branched alkyl end group in addition to an oxypropylene end group with from 1-5 groups. Contrary to common understanding, the surfactant is readily biodegradable with a branched alkyl end group. Further, despite conventional understanding of foaming and cleaning abilities based on order and degree of alkoxylation, the surfactant is both low foaming and an effective hard surface cleaner.

In a first aspect, the present invention is a surfactant having the following structure (I):

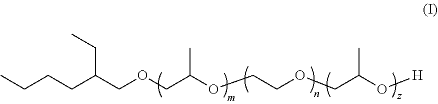

where m is a value in a range of 3 to 10, n is a value in a range of 3 to 20 and z is a value in a range of 1 to 5.

In a second aspect, the present invention is a method of using the surfactant of structure (I), the method comprising placing a detergent composition containing the surfactant in an automatic dishwasher, such as for example an automatic household dishwasher.

The present invention is useful as a low foaming non-ionic surfactant for applications such as cleaning solutions, including for example home and industrial and institutional automatic dishwasher, metal cleaning, bottle washing, window cleaning, floor cleaning, food and beverage processing, and other hard surface cleaning.

### DETAILED DESCRIPTION

As used herein, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

All ranges include endpoints unless otherwise stated. Parts per million (ppm) refers to weight parts based on total aqueous solution weight unless otherwise indicated. Subscript values in polymer formulae refer to mole average number of units per molecule for the designated component of the polymer.

Test methods refer to the most recent test method as of the priority date of this document unless a date is indicated with the test method number as a hyphenated two-digit number. References to test methods contain both a reference to the testing society and the test method number. Test method organizations are referenced by one of the following abbreviations: ASTM refers to ASTM International (formerly known as American Society for Testing and Materials); EN refers to European Norm; DIN refers to Deutsches Institut für Normung; and ISO refers to International Organization for Standards.

The surfactant of the present invention has the following structure (I).

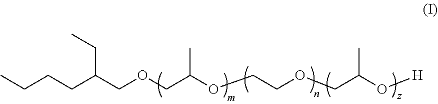

The variables “m” and “z” describe the average molar units of oxypropylene utilized in structure (I) and the variable “n” describes the average molar units of oxyethylene in structure (I). As defined herein, the m, n and z values are tested and determined by Proton Nuclear Magnetic Resonance Spectroscopy and Carbon-13 Nuclear Magnetic Resonance Spectroscopy. The m value of structure (I) is 3 or more, 4 or more, 5 or more, 6 or more, 7 or more, 8 or more, 9 or more, 10 or more, while at the same time 10 or less, 9 or less, 8 or less, 7 or less, 6 or less, 5 or less, 4 or less or 3 or less. For example, m may be from 3 to 10, or from 4 to 9, or from 5 to 9, or from 5 to 8, or from 5 to 7, or from 4 to 6. The n value of structure (I) is 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, while at the same time 20 or less, 19 or less, 18 or less, 17 or less, 16 or less, 15 or less, 14 or less, 13 or less, 12 or less, 11 or less, 10 or less, 9 or less, 8 or less, 7 or less, 6 or less, 5 or less, 4 or less or 3 or less. For example, n may be from 3 to 20, or from 3 to 9, or from 5 to 15, or 14 to 20. The z value of structure (I) is 1 or more, 2 or more, 3 or more, 4 or more, 5 more, while at the same time 5 or less, 4 or less, 3 or less, 2 or less, or 1 or less. For example, z may be from 1 to 5, or from 1 to 4, or from 2 to 4, or from 2 to 3. In a first specific example of structure (I), m is a value in a range of 3 to 10, n is a value in a range of 3 to 20 and z is a value in a range of 1 to 5. In a second specific example of structure (I), m is 5, n is in a range of 3 to 9 and z is in a range of 1 to 5. In a third specific example of structure (I), m is 5, n is in a range of 3 to 9 and z is in a range of 2 to 4. In a fourth specific example of structure (I), m is 5, n is in a range of 3 to 9 and z is in a range of 2 to 3.

The surfactant has a 2-ethylhexyl (2EH) moiety on one end and a hydroxyl moiety on the other end. The 2EH moiety is a branched alkyl with each branch having a length of two carbons or more. The 2EH end group moiety can be introduced into the molecule by using 2-ethylhexanol as an initiator to polymerize the blocks of oxypropylene and oxyethylene. Despite having a branched alkyl end group, the present surfactant is biodegradable. This is an unexpected result based on prior art teachings that explain having a branched alkyl detrimentally affects biodegradability. Surprisingly, the present surfactant having a branched alkyl end group is biodegradable when in an oxypropylene/oxyethylene/oxypropylene triblock structure. The surfactant of the present invention is also particularly good at defoaming. For example, it is surprisingly found that when n is 6 and z is 5 or less, for example 3, a surfactant of structure (I) has very low foaming at 23° C. while allowing a cloud point in water higher than 30° C. Further, such a surfactant is more efficient in removing greasy soils from a hard surface than a surfactant of structure (I) in which the z value is higher than 5.

The surfactant of the present disclosure is useful as a component in a fully formulated detergent in hard surface cleaning formulations, such as dishwashing detergents for automatic dishwashers and as a degreaser in industrial metal cleaning. To use the surfactant of the present disclosure as a dishwasher detergent, place the detergent composition containing the surfactant into an automatic dishwasher. To use the surfactant of the present disclosure as a metal cleaning detergent, place the detergent composition containing the surfactant in contact with a metal. The surfactant of the present disclosure has a cloud point of 23° C. or more, 30° C. or more, 35° C. or more and as such may be beneficial for addition into detergents for the applications outlined above.

### Examples

Prepare seven different surfactants (e.g., Examples 1-7) of structure (I) as described in Table 1 using the following procedure.

Charge 3339.5 grams of 2-ethylhexanol and 97.00 grams of 45% potassium hydroxide aqueous solution into a twenty liter reactor that has been purged with nitrogen. Gradually apply vacuum to the reactor over two hours to achieve 100 millimeter mercury. Remove 15.8 grams of mixture from the reactor and measure for water content by Karl Fisher titration (411 parts per million by weight (ppm)). Pressurize and vent the reactor seven times with dry nitrogen to remove atmospheric oxygen and pressurize with nitrogen to 110 to 139 kiloPascals (kPa) at 25° C. Heat the contents of the reactor while agitating to 145° C. and then meter in 8070 grams propylene oxide over 4 hours. After completing the propylene oxide feed, agitate the reactor contents at 145° C. for an additional 2 hours and then cool to 60° C. Remove 489.97 grams of reactor contents. Heat the reactor contents to 145° C. and meter in 6840 grams of ethylene oxide into the reactor over 4 hours. After completing the ethylene oxide feed, agitate the reactor contents at 145° C. for 2 hours and then cool to 60° C. Remove 360.4 grams of the reactor contents. Heat the reactor contents to 145° C. and meter in 2785 grams of propylene oxide over 4 hours and then continue agitating at 145° C. for an additional 2 hours. Cool the reactor contents to 60° C.

Remove 2155.7 grams of the reactor contents and neutralize with acetic acid to achieve a pH of 4-8 (in 10% aqueous solution) to obtain Example 1.

Heat the reactor contents back to 145° C. and meter in 1510 g of propylene oxide into the reactor over 4 hours. Continue agitating at 145° C. for an additional 2 hours and then cool to 60° C. Remove 3410.0 grams of reactor contents and neutralize with acetic acid in a 10% aqueous solution to a pH of 4-8 to obtain Example 2.

Heat the reactor contents back to 145° C. and meter in 2210 grams of propylene oxide over 4 hours and then continue to agitate for an additional 2 hours at 145° C. Cool the reactor contents to 60° C. Remove 1955.2 grams of reactor contents and neutralize with acetic acid in a 10% aqueous solution to a pH of 4-8 to obtain Example 3.

Examples 4-7 are prepared in like manner by adjusting the amount of propylene oxide and ethylene oxide feeds to the appropriate mole ratios for those Examples.

Determine the cloud point for Examples 1-7 with a one weight-percent (wt %) solution of the example in deionized water using a Mettler Toledo FP900 ThermalSystem with an FP90 central processor and FP81 measuring cell according to ASTM D2024-09.

Determine the Draves Wetting values for Examples 1-7 according to ASTM D2281-69. The results are the minimum concentration (in wt %) required to wet the tested skein in 20 seconds. Lower values correspond to better wetting ability for a surfactant.

Determine the contact angle at 21-23° C. using a Kruss DSA-100 Drop Shape Analyzer with a movable sample stage and Kruss software DSA3.exe to control operation of the instrument and perform data analysis. Measure the contact angle on a static sessile drop on a parafilm substrate. Place a parafilm on a glass microscope slide using a small amount of adhesive on each edge of the slide to hold the film in place. Place the substrate on a sample stage and deposit five liquid drops of a 0.1 wt % solution of surfactant in deionized water on the substrate programmatically using the procedure predefined via DSA software. Use a drop volume of five microliters. The rate of drop deposition is six microliters per minute and drop measurements were made immediately after drop placement. Once a drop is in place, an image of the drop is collected. Determine the baseline and left and right contact angles by software and determine the arithmetic mean of left and right contact angles for each drop. The result is a mean of the values from three groups of five drops (mean of 15 total drops).

Determine the surface tension of the surfactant using a 0.1 wt % aqueous surfactant solution and a Kruss D12 tensiometer fitted with a Wilhelmy platinum plate at 25° C. Make solutions by dissolving surfactant into deionized water. The surface tension of the deionized water to make the solutions is 72-73 milliNewtons per meter. The results are a mean of five repeated testing values with the standard deviation being less than 0.1 mN/m.

Determination of biodegradability is performed in accordance to Organization for Economic Cooperation and Development (OECD) test method 301F. Determination of Aquatic toxicity (A-tox) in milligrams per liter (mg/L) is performed according to OECD Guidelines for the Testing of Chemicals, “Daphnia sp., Acute Immobilization Test”, Test Guideline 202, adopted 13 Apr. 2004.

The properties of each of the surfactant Examples are included in Table 1. Each surfactant has the structure of structure (I) and the structure of each is given by specifying the values for m, n and z for each surfactant.

TABLE 1

| | | Cloud | Draves Wetting | | Surface | |
|---|---|---|---|---|---|---|
| | Structure | Point | 20s wetting | Contact | Tension | Biodegradability |
| Example | (m, n, z) | (° C.) | concentration (wt %) | Angle (°) | (dynes/cm) | (%) |
| 1 | 5, 6, 0 | 56 | 0.07 | 67 | 30.7 | 90 |
| 2 | 5, 6, 2 | 40 | 0.07 | 67 | 31.6 | 86 |
| 3 | 5, 6, 3 | 38 | 0.11 | 63 | 31.9 | 86 |
| 4 | 5, 6, 5 | 34 | 0.11 | 64 | 32.4 | N/A |
| Comparative | 5, 6, 7 | 14 | 0.12 | 68 | 33.1 | N/A |
| Example A | | | | | | |
| Comparative | 5, 6, 9 | <10 | 0.12 | 69 | 33.9 | N/A |
| Example B | | | | | | |
| Comparative | 5, 6, 10 | <10 | 0.18 | 65 | 34.1 | N/A |
| Example C | | | | | | |

Examples 1, 2 and 3 demonstrate a biodegradability value that is 80% or higher. A value of 60% is deemed “readily biodegradable” under the test method outlined above. Therefore, each of the Examples tested is deemed readily biodegradable. Example 4 and Comparative Examples A-C are not tested for biodegradability.

Foaming Properties

Foaming properties of Examples 1-4 and Comparative Examples A-C are tested by two methods, a Ross-Miles foam test following the guideline of ASTM D1173-53 and Waring Blender foam test. In the Waring Blender foam test, 200 ml of a surfactant solution in deionized water at 0.1 wt % concentration is added in a 1-liter container of a Waring™ Laboratory Blender (Model 31DM33, from Waring Commercial). The base solution volume is recorded. The blender is then turned on at high speed for 60 seconds to agitate the solution. The blender is stopped and the total volume of the solution and foam is recorded at 0 seconds, 30 seconds and 90 seconds after stopping of the blender.

Examples 1, 3, 4 and Comparative Example C were first tested by the Ross-Miles method, however none of the samples generated measurable foam height. The Waring Blender foam test method was then applied to all the Examples in Table 1 and the results were summarized in Table 2.

TABLE 2

| | | Volume at | Volume at | Volume at |
|---|---|---|---|---|
| Example | Base Volume | 0 Seconds | 30 Seconds | 90 Seconds |
| 1 | 200 ml | 325 ml | 250 ml | 250 ml |
| 2 | 200 ml | 325 ml | 250 ml | 250 ml |
| 3 | 200 ml | 300 ml | 250 ml | 250 ml |
| 4 | 200 ml | 250 ml | 225 ml | 225 ml |
| Comparative | 200 ml | 200 ml | 200 ml | 200 ml |
| Example A | | | | |
| Comparative | 200 ml | 200 ml | 200 ml | 200 ml |
| Example B | | | | |
| Comparative | 200 ml | 200 ml | 200 ml | 200 ml |
| Example C | | | | |

As seen in Table 2, as the z value reaches 2-5, the production and retention of foam is greatly reduced while the cloud points are above 30° C.

Example 3 is compared with commercial low foam surfactant products (e.g., Comparative Examples) that have similar cloud points using the Ross-Miles foam test method as outlined above. The results are summarized in Table 3. The cloud point data in Table 3 is determined with a one weight-percent (wt %) solution of sample in deionized water using a Mettler Toledo FP900 ThermalSystem with an FP90 central processor and FP81 measuring cell according to ASTM D2024-09.

TABLE 3

| | Cloud | Foam Height | | |
|---|---|---|---|---|
| | Point | (mm) | | |
| Sample | (° C.) | Initial | 5 min. | Chemistry* |
| Example 3 | 38 | 0 | 0 | Structure I |
| Comparative | 35 | 84 | 30 | Butoxylated and ethoxylated C8-C9 alcohols (for example |
| Example D | | | | Plurafac ® LF-221 nonionic surfactant available from BASF ®) |
| Comparative | 32 | 92 | 15 | Alkoxylated straight chain alcohols (for example Plurafac ® |
| Example E | | | | LF-400 nonionic surfactant available from BASF ®) |
| Comparative | 38 | 61 | 10 | 2-Propylheptanol-oxyethylene-propylene oxide copolymer (for |
| Example F | | | | example Plurafac ® LF-901 nonionic surfactant available from |
| | | | | BASF ®) |
| Comparative | 36 | 103 | 11 | Ethoxylated and propoxylated C8-C18 alcohols (for example |
| Example G | | | | Surfonic ® LF-17 nonionic surfactant available from |
| | | | | Huntsman ®) |
| Comparative | 43 | 95 | 28 | Ethoxylated and propoxylated linear alcohols (for example |
| Example H | | | | Nonidet ® SF-5 nonionic surfactant available from Evonic ®) |
| Comparative | 35 | 105 | 9 | Ethoxylated and-propoxylated fatty alcohols (for example |
| Example I | | | | Antarox ® FM-33 nonionic surfactant available from Solvay ®) |
| Comparative | 36 | 72 | 13 | Capped ethoxylated alcohol (for example Triton ® DF-16 |
| Example J | | | | nonionic surfactant available from The Dow Chemical |
| | | | | Company ®) |
| Comparative | 40 | 116 | 27 | Ethoxylated and propoxylated C12-C14 alcohol (for example |
| Example K | | | | TERGITOL ™ MINFOAM 1X nomonic surfactant available |
| | | | | from The Dow Chemical Company ®) |

As can be seen from the data in Table 3, Example 3 shows clear low foam advantage over all the listed competitive products while maintaining a high cloud point.

Hard Surface Cleaning Properties

Removing oily soil from hard surfaces, such as vinyl tiles, is facilitated by a surfactant. A conventional industry test to evaluate hard surface cleaning efficiency is the Gardner Scrub Test (ASTM D-2486). A high throughput hard surface cleaning efficiency test following the ASTM D-2486 method is used to evaluate the hard surface cleaning efficiency of Examples 1-4 along with the Comparative Examples A-C. The level of cleaning is determined by the “Grey value” of the scrubbed spot after the cleaning. The higher the Grey value, the whiter the scrubbed spot is (i.e., because more of the oily soil has been removed) and the better the cleaning efficiency. The level of cleaning can also be compared by direct visual observation to the whiteness of the scrubbed spot after cleaning.

The hard surface Gardner Scrub Test is performed by creating a formulation of each of Examples 1-4 and the Comparative Examples A-K. Each of the formulations included 1 wt % of one of Examples 1-4 or Comparative Examples A-K, 3 wt % DOWANOL™ propylene glycol n-butyl ether (available from Dow Chemical), 0.5 wt % Monoethanolamine (MEA) (available from Sigma-Aldrich) and 95.5 wt % deionized water. Each of the formulations was a stable clear solution.

The Grey values from the hard surface cleaning evaluation for the formulations of Examples 1-7 are provided in Table 4.

TABLE 4

| Example | Grey Value |
|---|---|
| 1 | 219.4 ± 3.4 |
| 2 | 219.1 ± 4.8 |
| 3 | 218.1 ± 4.8 |
| 4 | 208.3 ± 3.4 |
| Comparative Example A | 208.0 ± 3.4 |
| Comparative Example B | 204.4 ± 3.4 |
| Comparative Example C | 202.8 ± 3.4 |
| Water | 132.4 ± 2.4 |

As can be seen from the data of Table 4, with the increasing size of terminal oxypropylene blocks, the degreasing efficiency of Examples 1-4 and Comparative Examples A-C is decreased. However, when the z value of Structure (I) was controlled to about 5 or less, the degreasing efficiency was greatest.

Example 3 was compared with the Comparative Examples of Table 3 for hard surface cleaning. Table 5 provides the Grey values for the Example 3 as compared to the Comparative Examples D-K.

TABLE 5

| Sample | Grey Value |
|---|---|
| Example 3 | 213.2 ± 2.2 |
| Comparative Example D | 160.6 ± 2.7 |
| Comparative Example E | 192.4 ± 2.7 |
| Comparative Example F | 190.4 ± 2.7 |
| Comparative Example G | 156.1 ± 2.7 |
| Comparative Example H | 160.3 ± 2.7 |
| Comparative Example I | 167.3 ± 2.7 |
| Comparative Example J | 173.1 ± 2.7 |
| Comparative Example K | 197.6 ± 2.7 |
| Water | 127.8 ± 3.9 |

As can be seen from Table 5, Example 3 demonstrated significantly better cleaning efficiency than all the Comparative Examples.

Metal Cleaning Performance

Testing the metal cleaning performance of Examples 1-4 and Comparative Examples A-C is performed with a modified method of JB/T 4323.2-1999 “Test methods of water-based metal cleanser.” The following steps are performed for the testing of each Example:

1) An “oil soil” mix is prepared by mixing 2 parts N32 HL machinery oil, 1 part Vaseline and 1 part Barium petroleum sulfonate at 120° C. The oil soli mix is then cooled to temperature for use.

2) Polished 45# steel plates (40 mm×13 mm×2 mm) are cleaned with petroleum naphtha and ethanol with an absorbent cotton and then dried in a dryer.

4) A thin layer of the oil soil is applied to each of the plates using a glass rod. Excess oil soil on the edges of the steel plate is wiped with a tissue paper. The total oil soil weight on the steel plate is controlled within 50 mg to 60 mg per plate.

5) Different detergent formulations are prepared with 3 wt % Sodium Tripolyphosphate (available from Sigma-Aldrich), 2 wt % Sodium Metasilicate (available from Sigma-Aldrich), 0.5 wt % EDTA-4Na (available from Sigma-Aldrich), 5 wt % of a surfactant and the balance was water. The detergent is then diluted with deionized water to a 1:19 ratio (by weight) for use.

6) Each oil soil coated steel plate is placed into separate detergent solutions and ultrasonic energy is applied in the detergent solution for approximately 10 seconds. Each plate is removed and placed in deionized water for 1 to 2 seconds for rinsing purposes. The steel plate surfaces are visually checked for residue of oil soil. If the steel plate surface is free of oil soil, the cleaning time is recorded as 10 seconds. If oil soil still exists on the surface of the steel plate, the cycle of ultrasonic cleaning and rinsing is repeated until the plate surface is free of oil soil. The total ultrasonic and rinsing time is recorded as the cleaning time.

As both Example 3 and Comparative Example F have cloud points greater than 23° C., two temperatures ranges are used in the metal cleaning tests. A lower temperature range of 20° C. to 25° C. and a higher temperature range of 35° C. to 40° C. are selected as these temperature ranges are close to the cloud points of Example 3 and Comparative Example F. The results of the metal cleaning testing are provided in Table 6.

TABLE 6

| | Temperature Range | Cleaning Time |
|---|---|---|
| Sample | (° C.) | (s) |
| Comparative Example F | 20-25 | 30-40 |
| Example 3 | 20-25 | 150 |
| Comparative Example F | 35-40 | 120 |
| Example 3 | 35-40 | 40-50 |

As evident from Table 6, the overall cleaning performance of Comparative Example F is better than Example 3 at the lower temperature range of 20° C. to 25° C. When the cleaning temperature is increased to 35-40° C., Example 3 requires less cleaning time than Comparative Example F. As such, although Comparative Example F and Example 3 have comparable cloud points, Example 3 exhibits better metal surface abilities at elevated temperatures than Comparative Example F.

1. A surfactant having the following structure (I): 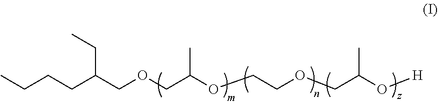
where m is a value in a range of 3 to 10, n is a value in a range of 3 to 20 and z is a value in a range of 1 to 5.

2. The surfactant of claim 1, further characterized by m being 5, n being in a range of 3 to 9 and z being in a range of 1 to 5.

3. The surfactant of claim 2 further characterized by m being 5, n being in a range of 3 to 9 and z being in a range of 2 to 4.

4. The surfactant of claim 1, further characterized by m being 5, n being in a range of 3 to 9 and z being in a range of 2 to 3.

5. A method of using the surfactant of claim 1, the method comprising placing a detergent composition containing the surfactant in contact with a metal.
